(19) United States
(12) Patent Application Publication
(10) Pub. No.: US 2006/0165814 A1
(43) Pub. Date: Jul. 27, 2006

(54) Amines, combination of amines and vanadium and amine vanadium salts for the treatment or prevention of dyslipidemia

(75) Inventors: Alec Mian, Barcelona (ES); Antonio Zorzano Olarte, Barcelona (ES); Luc Marti Clauzel, Barcelona (ES)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Genmedica Therapeutics SL, Barcelona (ES)

(21) Appl. No.: 11/303,171

(22) Filed: Dec. 16, 2005

Related U.S. Application Data
(60) Provisional application No. 60/636,821, filed on Dec. 16, 2004.

(51) Int. Cl. A61K 33/26; A61K 31/4706; A61K 31/4172; A61K 31/405; A61K 31/137; A61K 31/135; A61K 31/13

(52) U.S. Cl. 424/646; 514/649; 514/657; 514/663; 514/673; 514/400; 514/419; 514/313

(57) ABSTRACT

Combinations comprising vanadium (IV)/(V) compounds and pharmaceutically acceptable amines are provided. Combinations thereof are provided for the treatment or prevention of dyslipidemia, diabetes, obesity, metabolic syndrome or cardiovascular diseases.

### FIELD OF THE INVENTION

This invention relates to pharmaceutical compositions comprising amines, a combination of an amine and pharmaceutically acceptable vanadium compounds or amine vanadium salts, for use as dyslipidemia agents.

### BACKGROUND OF THE RELATED ART

Dyslipidemia is a generic name for a variety of disorders of blood lipid content that are associated with increased risk of coronary heart disease (CHD), particularly in patients with type 2 diabetes melitus. Dyslipidemia is associated with serum triglyceride elevation, which can lead to pancreatitis; LDL cholesterol elevation; or elevation in both LDL cholesterol and triglycerides. The condition results in increase in the risk of atherosclerotic diseases, including coronary heart disease, peripheral arterial disease, and cerebrovascular disease. Dyslipidemia is also associated with conditions characterized by insulin resistance and represents one of the key features of the Type 2 diabetes metabolic syndrome. In fact, it is thought that insulin resistance may lead to dyslipidemia and this is responsible for the metabolic abnormalities that cause much of the increased cardiovascular risk of subjects with metabolic syndrome.

In this regard, there is strong emphasis on the importance of achieving lipid profile targets; for example, in Europe these are a total cholesterol of 5 mmol/L and a low density lipoprotein (LDL) cholesterol of 3 mmol/L, respectively, while in the United States the targets are total cholesterol less than 200 mg/dL and LDL less than 100 mg/dL. However, there is a continued need in the art to improve the confidence that treatment to these target values makes best use of limited healthcare resources.

In recent years, several inorganic compounds have been described which mimic the effects of insulin, both in vivo and in isolated cells and tissues, and have been evaluated for their capacity to overcome type 2 diabetes-associated insulin resistance. These include vanadium (IV)/(V) compounds (cf. Heyliger et al., “Effect of vanadate on elevated blood glucose and depressed cardiac performance of diabetic rats”, Science 1985, vol. 227, pp. 1474-7); selenate (cf. McNeill et al., “Insulin-like effects of sodium selenate in streptozotrocin-induced diabetic rats”, Diabetes 1991, vol. 40, pp. 1675-8), lithium salts (cf. Rodriguez-Gil et al., “Lithium restores glycogen synthesis from glucose in hepatocytes from diabetic rats”, Arch. Biochem. Biophys. 1993, vol. 301, pp. 411-5), and tungsten (VI) compounds (cf. U.S. Pat. No. 5,595,763).

Among the vanadium derivatives which have been studied as insulin-mimickers are: vanadates and peroxovanadium complexes (vanadium in its +5 oxidation state combined with oxygen, specially orthovanadate VO₄³⁻, cf. U.S. Pat. No. 4,882,171), and vanadyl VO²⁺ salts and complexes (vanadium in its +4 oxidation state; cf. U.S. Pat. No. 5,300,496). The present inventors have shown previously that some combinations of amines such as benzylamine or tyramine and low concentrations of vanadate stimulates glucose transport in rat adipocytes (see Enrique-Tarancon et al., 1998, J. Biol. Chem. 273:8025-8032; Marti et al., 1998, J. Pharmacol. Exp. Ther. 285:342-349). In addition, chronic treatment with benzylamine and vanadate exerts potent antidiabetic effects in streptozotocin-induced diabetic rats (Marti et al., 2001, Diabetes 50: 2061-2068) or in Goto-Kakizaki diabetic rats (Abella et al., 2003 Diabetes 52: 1004-1013).

The use of vanadium compounds has as a principale drawback that it is toxic at therapeutically-effective doses. Administered concentrations must be close to the toxic level in order to achieve insulin-mimetic effects in animals. Vanadium treatment is always accompanied by marked negative side effects that are independent of the chemical form of vanadium used (Domingo et al., 1991, “Oral vanadium administration to streptozocin-diabetic rats has marked negative side-effects wich are independent of the form of vanadium used”, Toxicology 66: 279-87). Remarkable signs of vanadium compounds toxicity, including significant mortality, are observed at all doses capable of lowering blood glucose. It is expected that vanadium compound concentrations effective as insulin mimickers also will be necessary to achieve ameliorating or alleviating effects on dyslipidemia.

Thus, there is a pressing need in the art to develop formulations of vanadium compounds sufficient to achieve a substantial reduction in doses of vanadium compounds, while keeping their insulin-mimicker activity, sufficient to achieve concomitant reduction of dyslipidemia.

### SUMMARY OF THE INVENTION

This invention provides pharmaceutical compositions and formulations comprising a pharmaceutically acceptable vanadium (IV)/(V) compound and a pharmaceutically acceptable amine that is a substrate of semicarbazide-sensitive amine oxidase (SSAO) or other copper-containing amine oxidase, in admixture with pharmaceutically acceptable excipients or carriers. Said compositions and formulations are useful for treating and/or preventing dyslipidemia in mammals, particularly humans. In certain embodiments, dyslipidemia is associated with Type 1 or Type 2 Diabetes mellitus. As provided herein, the active ingredients of the combination comprising the compositions or formulations are administered simultaneously, separatedly or sequentially. The compositions or formulations of the invention can be administered parenterally, or by oral administration which is preferred.

In preferred embodiments, the pharmaceutical combination or formulation comprises a vanadium compound that is a vanadate, peroxovanadium complex, vanadyl salt or vanadyl complex. Specially preferred embodiments are pharmaceutical combinations or formulations comprising a vanadate, and more preferred those comprising sodium orthovanadate Na₃VO₄.

In preferred embodiments, the pharmaceutical compositions and formulations comprise an amine that is tyramine, benzylamine, 3-phenyl-propylamine, 2-(4-fluoro-phenyl)-ethylamine, 4-phenyl-butylamine, 4-fluoro-benzylamine, 2,3-dimethoxybenzylamine, 1-naphtalenemethylamine, deoxyepinephrine, epinephrine, norepinephrine, dopamine, histamine, β-phenylethylamine, N-acetylputrescine, tryptamine, n-octylamine, n-pentylamine, kynuramine, 3-methoxytyramine, or n-decylamine, hexylethanolamine, octopamine, spermine, spermidine, N-acetylspermine, or N-acetylspermidine

Specially preferred are combinations and formulations comprising benzylamine, 3-phenyl-propylamine, 2-(4-fluoro-phenyl)-ethylamine, 4-phenyl-butylamine, 4-fluoro-benzylamine, 2,3-dimethoxybenzylamine, or 1-naphtalenemethylamine. The amine can also be in the form of a salt with any of the pharmaceutically acceptable organic or inorganic acids known in the art. Specific preferred amines are substrates of semicarbazide-sensitive amine oxidase or other copper-containing amine oxidase as disclosed in co-owned and copending U.S. Ser. No. 60/598,010, filed Aug. 2, 2004.

Further advantageous amines for use in the practice of this invention are disclosed in co-owned and co-pending U.S. Ser. No. 10/430,235, published as No. US 2004-0224031 A1, 11 Nov. 2004.

Another aspect of the present invention relates to methods for treating and/or preventing dyslipidemia in a mammal, specially in a human, comprising the step of administering a pharmaceutical combination or formulation of the invention. Use of a mixture of a pharmaceutically acceptable vanadium (IV)/(V) compound and a pharmaceutically acceptable amine, or a salt thereof, for preparing a medicament for treating and/or preventing dyslipidemia is also part of the present invention.

Specific preferred embodiments of the present invention will become evident from the following more detailed description of certain preferred embodiments and the claims.

### BRIEF DESCRIPTION OF THE DRAWINGS

The results are, in part, set out graphically in the following figures:

FIG. 1 is a bar graph of the plasma concentration of cholesterol (Ch) (expressed in mmol/L) measured in nondiabetic rats (C), streptozotocin-induced diabetic rats (D) or diabetic rats treated for 17 days with a single daily oral dose of hexaquis(benzylammonium) decavanadate (5 μmol/kg/day between day 0 and day 7 and 10 μmol/kg/day from day 8 to day 17 of treatment) (BV). Cholesterol concentrations in the D group were statistically significant from the C group at p<0.05.

FIG. 2A though 2D are bar graphs showing plasma concentrations of insulin (FIG. 2A), glucose (FIG. 2B), free fatty acids (FIG. 2C) and triglycerides (FIG. 2D) measured in nondiabetic rats (C), streptozotocin-induced diabetic rats (Diab) or diabetic rats treated for 28 days with subcutaneously infused decanadate (V10, 1.25 μmol/kg/day) or hexaquis(benzylammonium) decavanadate (B6V10) at two different doses (1.25 μmol/kg/day or 2.5 μmol/kg/day). * indicates the existance of statistically significant differences from the Diab group at p<0.05.

FIG. 3A through 3D show plasma concentrations of total cholesterol (FIG. 3A), nonesterified cholesterol (FIG. 3B), esterified cholesterol (FIG. 3C) and HDL-cholesterol (FIG. 3D) measured in nondiabetic rats (C), streptozotocin-induced diabetic rats (Diab) or diabetic rats treated for 28 days with subcutaneously infused decanadate (V10, 1.25 μmol/kg/day) or hexaquis(benzylammonium) decavanadate (B6V10) at two different doses (1.25 μmol/kg/day or 2.5 μmol/kg/day). * indicates the existance of statistically significant differences from the Diab group at p<0.05.

### DETAILED DESCRIPTION OF PREFERRED EMBODIMENTS

This invention provides pharmaceutical compositions comprising combinations or formulations of a vanadium-containing compound and an amine for the treatment or prevention or both of dyslipidemia, particularly dyslipdemia associated with type 1 or type 2 diabetes melitus.

As provided herein, the term “vanadium-containing compound” is intended to encompass any organic or inorganic compound comprising a vanadium atom in any oxidation state. Particularly as provided herein, a vanadium compound comprises a vanadate, peroxovanadium complex, vanadyl salt or vanadyl complex. Specially provided are pharmaceutical combinations or formulations comprising a vanadate, particularly sodium orthovanadate Na₃VO₄.

Specifically provided herein are vanadium (IV)/(V) compounds. As disclosed herein, the term “a pharmaceutically acceptable vanadium (IV)/(V) compound” is intended to include any chemical entity formed by one or several vanadium atoms in its +4 or its +5 oxidation states, attached to a chemical structure that is pharmaceutically acceptable by itself. The cations V⁴⁺ and V⁵⁺ have never been observed isolated, and they come always accompanied with a chemical moiety partially formed by a coordination sphere. The coordination sphere can be formed by inorganic ligands (oxide, hydroxide, peroxide, etc) as, for example, the orthovanadate anion VO₄³⁻ (vanadium +5 and a coordination sphere formed by four oxide ions), and the vanadyl cation VO²⁺ (vanadium +4 and a coordination sphere formed by one oxide ions). The coordination sphere can also be formed by organic ligands which are molecules or ions attached to the vanadium atoms through O, S or N atoms belonging to different pharmaceutically acceptable organic compounds (e.g. pharmaceutically acceptable alcohols, thiols, carboxilyc acids, amines, amino acids, N-containing heterocycles, etc). Mixed inorganic/organic coordination spheres are also possible as, for example, in peroxovanadium complex ions [VO(O₂)₂L-L′]ⁿ⁻, L-L′ being a bidentate organic ligand such as 1,10-phenanthroline. When the structure formed by the vanadium atom and its coordination sphere is not neutral, the term “chemical moiety” also includes any pharmaceutically-acceptable ionic species which makes neutral the whole compound. For example, vanadate anions are always accompanied by cations (e.g. sodium, potassium, magnesium, calcium) to form neutral salts, and said salts are also encompassed in the vanadium-containing compounds of the invention. The term “pharmaceutically acceptable vanadium (IV)/(V) compound” also include any pharmaceutically acceptable solvate (e.g. hydrate) of said compound.

As disclosed herein the effective concentrations of vanadate in the pharmaceutical compositions or combinations thereof are one order of magnitude lower (or less) than the concentrations needed when vanadate is administered alone. This means that there is synergism between the vanadium compound and the amine. From a practical point of view, such synergism means that the amount of the vanadium-containing compound administered, and hence the toxicity of the drug is much lower when the combination of amine+vanadium-containing compound is administered compared with administration of the vanadium-containing compound alone, to achieve a particular dyslipidemia-ameliorating effect. This represents an important advantage of the pharmaceutical combination of the present invention in respect of the vanadium-compositions known in the art, for the treatment and/or prevention of dyslipidemia.

As used herein, the term “amine” or “amine-containing compound” is intended to encompass any compound, preferably an organic compound containing a primary or substituted amine moiety. Specifically provided by the invention are primary amines, such as: tyramine, benzylamine, 3-phenyl-propylamine, 2-(4-fluoro-phenyl)-ethylamine, 4-phenyl-butylamine, 4-fluoro-benzylamine, 2,3-dimethoxybenzylamine, 1-naphtalenemethylamine, deoxyepinephrine, epinephrine, norepinephrine, dopamine, histamine, β-phenylethylamine, N-acetylputrescine, tryptamine, n-octylamine, n-pentylamine, kynuramine, 3-methoxytyramine, or n-decylamine, hexylethanolamine, octopamine, spermine, spermidine, N-acetylspermine, or N-acetylspermidine. Specific preferred amines are substrates of semicarbazide-sensitive amine oxidase or other copper-containing amine oxidase as disclosed in co-owned and copending U.S. Ser. No. 10/430,235, published as No. US 2004-0224031 A1, 11 Nov. 2004 and U.S. Ser. No. 60/598,010, filed Aug. 2, 2004.

Specific amines comprising the pharmaceutical compositions and formulations provided by this invention include compounds that are substrates of semicarbazide-sensitive amine oxidase or other copper-containing amine oxidase having the formula: 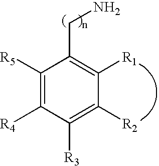
wherein
R₁, R₂, R₃, R₄ and R₅ are radicals that are independently H, OH, (C₁-C₆)-alkyl, (C₁-C₆)-alkoxy, NR₆R₇, (CH₂)ₚNR₈R₉, O(CH₂)_{q}Ph, CONR₁₀R₁₁, COR₁₂, CF₃, OCF₃, F, Cl, Br, NO₂, or CH₂NHC(═NH)NH₂; or alternatively R₁ and R₂ are bound together forming a ring with a fused benzene;
p and q are integers from 1 to 3;
R₆, R₇, R₈, R₉, R₁₀, R₁₁ and R₁₂ are radicals that are independently H, (C₁-C₄)-alkyl or aryl, particularly phenyl;
and where n is an integer from 1 to 3.

As provided herein, said amines are further advantageously administered simultaneously, separately or sequentially with a pharmaceutically-acceptable vanadium (IV)/(V) compound, preferably a vanadate, peroxovanadium complex, vanadyl salt or vanadyl complex. In certain embodiments said vanadium compound has the formula [HₓV₁₀O₂₈]^{y-}, where x is an integer from 0 to 2, and where y is an integer from 4 to 6, provided that x+y=6.

As used herein:

By “alkyl”, “C₁-C₆ alkyl”, and “C₁-C₆ alkyl” in the present invention is meant straight or branched chain alkyl groups having 1-6 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl.

By “alkoxy”, “C₁-C₆ alkoxy”, and “C₁-C₆ alkoxy” in the present invention is meant straight or branched chain alkoxy groups having 1-6 carbon atoms, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

By the term “halogen” in the present invention is meant fluorine, bromine, chlorine, and iodine.

By “cycloalkyl”, e.g., C₃-C₇ cycloalkyl, in the present invention is meant cycloalkyl groups having 3-7 atoms such as, for example cyclopropyl, cyclobutyl, cyclopentyl, cyclohexyl, and cycloheptyl.

By “aryl” is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl, anthryl, or phenanthryl), which is optionally mono-, di-, or trisubstituted with, e.g., halogen, C₁-C₆ alkyl, C₁-C₆ alkoxy, C₁-C₆ alkylthio, trifluoromethyl, C₁-C₆ acyloxy, aryl, heteroaryl, and hydroxy. Preferred aryl groups include phenyl, indanyl, biphenyl, and naphthyl, each of which is optionally substituted as defined herein. More preferred aryl groups include phenyl and naphthyl, each of which is optionally substituted as defined herein.

By “heteroaryl” is meant an aromatic ring or aromatic ring system, wherein each ring contains of 5-, 6-, or 7-members wherein at least one and up to four ring members are selected from nitrogen, oxygen, or sulfur. Such heteroaryl groups include, for example, thienyl, furanyl, thiazolyl, imidazolyl, (is)oxazolyl, pyridyl, pyrimidinyl, (iso)quinolinyl, indolyl, napthyridinyl, benzimidazolyl, and benzoxazolyl. Preferred heteroaryls are thiazolyl, pyrimidinyl, pyrimidin-2-yl, indolyl, pyridyl, 1-imidazolyl, 2-thienyl, 1-, or 2-quinolinyl, 1-, or 2-isoquinolinyl, 1-, or 2-tetrahydro isoquinolinyl, 2- or 3-furanyl and 2-tetrahydrofuranyl. By “heterocycloalkyl,” is meant one or more carbocyclic ring systems of 3, 4, 5, 6, or 7-membered rings which includes fused ring systems of 9-11 atoms containing at least one and up to four heteroatoms selected from nitrogen, oxygen, and sulfur. Preferred heterocycles of the present invention include morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, azepanyl, diazepanyl, tetrahydrothienyl S-oxide, tetrahydrothienyl S,S-dioxide and homothiomorpholinyl S-oxide.

As provided herein, the pharmaceutical composition comprising a vanadium-containing compound and an amine are provided as two separate compounds, a salt of the two or a single compound comprising both a vanadium-containing compound and an amine.

Compounds of the invention are useful as pharmaceutical agents, and can be provided as pharmaceutical compositions. The pharmaceutical compositions can be manufactured in a manner that is itself known, e.g., by means of a conventional mixing, dissolving, granulating, dragee-making, levigating, emulsifying, encapsulating, entrapping or lyophilizing processes.

Pharmaceutical compositions can be formulated in conventional manner using one or more physiologically acceptable carriers comprising excipients and auxiliaries that facilitate processing of the active compounds into preparations that can be used pharmaceutically. Proper formulation is dependent upon the route of administration chosen.

Non-toxic pharmaceutical salts include salts of acids such as hydrochloric, phosphoric, hydrobromic, sulfuric, sulfinic, formic, toluenesulfonic, methanesulfonic, nitic, benzoic, citric, tartaric, maleic, hydroiodic, alkanoic such as acetic, HOOC—(CH₂)ₙ—CH₃ where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

For injection, the compounds prepared according to the methods of the invention can be formulated in appropriate aqueous solutions, such as physiologically compatible buffers such as Hanks's solution, Ringer's solution, or physiological saline buffer. For transmucosal and transcutaneous administration, penetrants appropriate to the barrier to be permeated are used in the formulation. Such penetrants are generally known in the art.

For oral administration, the compounds can be formulated readily by combining the active compounds with pharmaceutically acceptable carriers well-known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of tablets or lozenges formulated in conventional manner.

For administration by inhalation, the compounds prepared according to the methods of the invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyloleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use. The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in acceptable oil) or ion exchange resins, or as sparingly soluble derivatives, for example, as a sparingly soluble salt.

A pharmaceutical carrier for hydrophobic compounds of formula I is a cosolvent system comprising benzyl alcohol, a nonpolar surfactant, a water-miscible organic polymer, and an aqueous phase. The cosolvent system can be the VPD co-solvent system. VPD is a solution of 3% w/v benzyl alcohol, 8% w/v of the nonpolar surfactant polysorbate 80, and 65% w/v polyethylene glycoL300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of polysorbate 80; the fraction size of polyethylene glycol can be varied; other biocompatible polymers can replace polyethylene glycol, e.g. polyvinyl pyrrolidone; and other sugars or polysaccharides can substitute for dextrose.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also can be employed, although usually at the cost of greater toxicity. Additionally, the compounds can be delivered using a sustained-release system, such as semipermeable matrices of solid hydrophobic polymers containing the therapeutic agent. Various sustained-release materials have been established and are well known by those skilled in the art. Sustained-release capsules can, depending on their chemical nature, release the compounds for a few weeks up to over 100 days. Depending on the chemical nature and the biological stability of the therapeutic reagent, additional strategies for protein and nucleic acid stabilization can be employed.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The compounds of the invention can be provided as salts with pharmaceutically compatible counterions. Pharmaceutically compatible salts can be formed with many acids, including but not limited to hydrochloric, sulfuric, acetic, lactic, tartaric, malic, succinic, phosphoric, hydrobromic, sulfinic, formic, toluenesulfonic, methanesulfonic, nitic, benzoic, citric, tartaric, maleic, hydroiodic, alkanoic such as acetic, HOOC—(CH₂)ₙ—CH₃ where n is 0-4, and the like. Salts tend to be more soluble in aqueous or other protonic solvents that are the corresponding free base forms. Non-toxic pharmaceutical base addition salts include salts of bases such as sodium, potassium, calcium, ammonium, and the like. Those skilled in the art will recognize a wide variety of non-toxic pharmaceutically acceptable addition salts.

Pharmaceutical compositions of the compounds prepared according to the methods of the invention can be formulated and administered through a variety of means, including systemic, localized, or topical administration. Techniques for formulation and administration can be found in “Remington's Pharmaceutical Sciences,” Mack Publishing Co., Easton, Pa. The mode of administration can be selected to maximize delivery to a desired target site in the body. Suitable routes of administration can, for example, include oral, rectal, transmucosal, transcutaneous, or intestinal administration; potential delivery, including intramuscular, subcutaneous, intramedullary injections, as well as intrathecal, direct intraventricular, intravenous, intraperitoneal, intranasal, or intraocular injections.

Alternatively, one can administer the compound in a local rather than systemic manner, for example, via injection of the compound directly into a specific tissue, often in a depot or sustained release formulation.

Pharmaceutical compositions suitable for use include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For administration to non-human animals, the drug or a pharmaceutical composition containing the drug may also be added to the animal feed or drinking water. It will be convenient to formulate animal feed and drinking water products with a predetermined dose of the drug so that the animal takes in an appropriate quantity of the drug along with its diet. It will also be convenient to add a premix containing the drug to the feed or drinking water approximately immediately prior to consumption by the animal.

Preferred compounds prepared according to the methods of the invention will have certain pharmacological properties. Such properties include, but are not limited to oral bioavailability, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Serum protein binding may be predicted from albumin binding assays. Such assays are described in a review by Oravcová et al. (1996, Journal of Chromatography B-Biomedical Applications 677:1-28). Compound half-life is inversely proportional to the frequency of dosage of a compound. In vitro half-lives of compounds may be predicted from assays of microsomal half-life as described by Kuhnz and Gieschen (1998, Drug Metabolism and Disposition 26:1120-1127).

Toxicity and therapeutic efficacy of such compounds can be determined by conventional pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD50 and ED50. Compounds that exhibit high therapeutic indices are preferred. The data obtained from these cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. The exact formulation, route of administration and dosage can be chosen by the individual physician in view of the patient's condition. (See, e.g. Fing et al., 1975, in THE PHARMACOLOGICAL BASIS OF THERAPEUTICS, Ch. 1, p. 1).

Dosage amount and interval can be adjusted individually to provide plasma levels of the active moiety that are sufficient to maintain bacterial cell growth-inhibitory effects. Usual patient dosages for systemic administration range from 1-200 mg/day. Stated in terms of patient body surface areas, usual dosages range from 50-910 mg/m²/day, more preferably from 0.06-0.25 mg/kg/day or 2 to 40 mg/day. Usual average plasma levels should be maintained within 0.1-1000 μM

In cases of local administration or selective uptake, the effective local concentration of the compound cannot be related to plasma concentration.

Compounds provided by the present invention are useful the treatment or prevention of dyslipidemia, whether or not associated with Type 1 or Type 2 diabetes, or associated with metabolic syndrome.

Prophylaxis is expected to be particularly relevant to the treatment of persons who have suffered a previous episode of, or are otherwise considered to be at increased risk of, dyslipidemia or Type 1 or Type 2 diabetes. Persons at risk of developing a particular disease or condition generally include those having a family history of the disease or condition, or those who have been identified by genetic testing or screening to be particularly susceptible to developing the disease or condition.

As used herein, the terms “treating” or “treatment” of a disease includes: (1) preventing the disease, i.e. causing the clinical symptoms of the disease not to develop in a mammal that may be exposed to or predisposed to the disease but does not yet experience or display symptoms of the disease, (2) inhibiting the disease, i.e., arresting or reducing the development of the disease or its clinical symptoms, or (3) relieving the disease, i.e., causing regression of the disease or its clinical symptoms.

As used herein, the term “therapeutically effective amount” means the amount of a compound that, when administered to a mammal for treating a disease, is sufficient to effect such treatment for the disease. The “therapeutically effective amount” will vary depending on the compound, the disease and its severity and the age, weight, or other relevant characteristics of the mammal to be treated.

The compounds of the present invention may be prepared by use of known chemical reactions and procedures. Representative methods for synthesizing compounds of the invention are presented below. It is understood that the nature of the substituents required for the desired target compound often determines the preferred method of synthesis. All variable groups of these methods are as described in the generic description if they are not specifically defined below.

Representative compounds prepared according to the methods of the present invention include, but are not limited to the compounds disclosed herein and their pharmaceutically acceptable acid and base addition salts. In addition, if a compound is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds.

The disclosures in this application of all articles and references, including patents, are incorporated herein by reference.

The following Examples are provided for the purposes of illustration and are not intended to limit the scope of the present invention. The present invention is not to be limited in scope by the exemplified embodiments, which are intended as illustrations of individual aspects of the invention. Indeed, various modifications of the invention in addition to those shown and described herein will become apparent to those skilled in the art from the foregoing description and accompanying drawings. Such modifications are intended to fall within the scope of the appended claims.

### EXAMPLES

### Example 1

### Effect of Oral and Chronic Administration of Hexaquis(benzylammonium) Decavanadate on Plasma Cholesterol Levels in Diabetic Rats

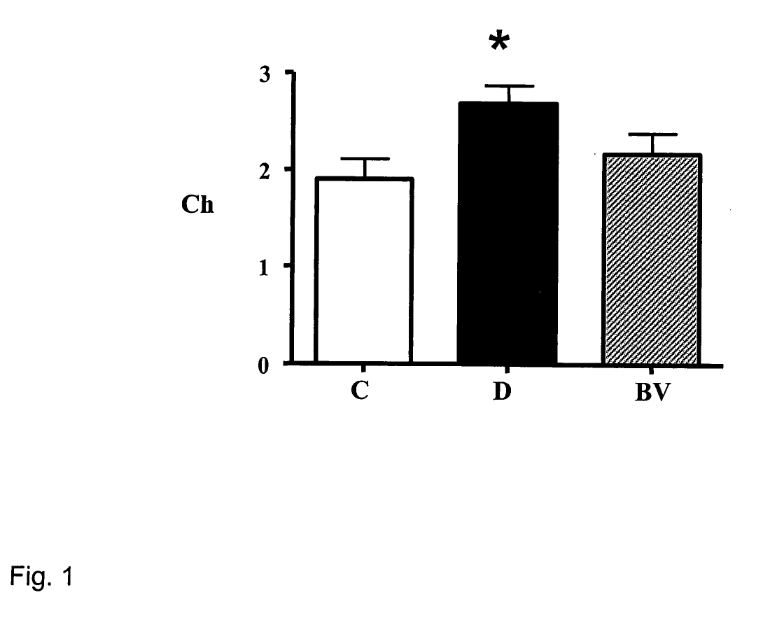

Oral administration of hexaquis(benzylammonium) decavanadate was tested to determine whether it ameliorated dyslipidemia associated to diabetes. Diabetes was induced in rats by intravenous administration of streptozotocin, causing a substantial increase in plasma cholesterol concentrations compared to nondiabetic rats (FIG. 1). Under these conditions, oral treatment with a daily dose of hexaquis(benzylammonium) decavanadate substantially reduced plasma cholesterol levels so that no differences between control and hexaquis(benzylammonium) decavanadate-treated rats were detectable.

### Example 2

### Effect of Subcutaneous and Chronic Administration of Hexaquis(benzylammonium) Decavanadate on Plasma Triglyceride and Free Fatty Acids Levels in Diabetic Rats

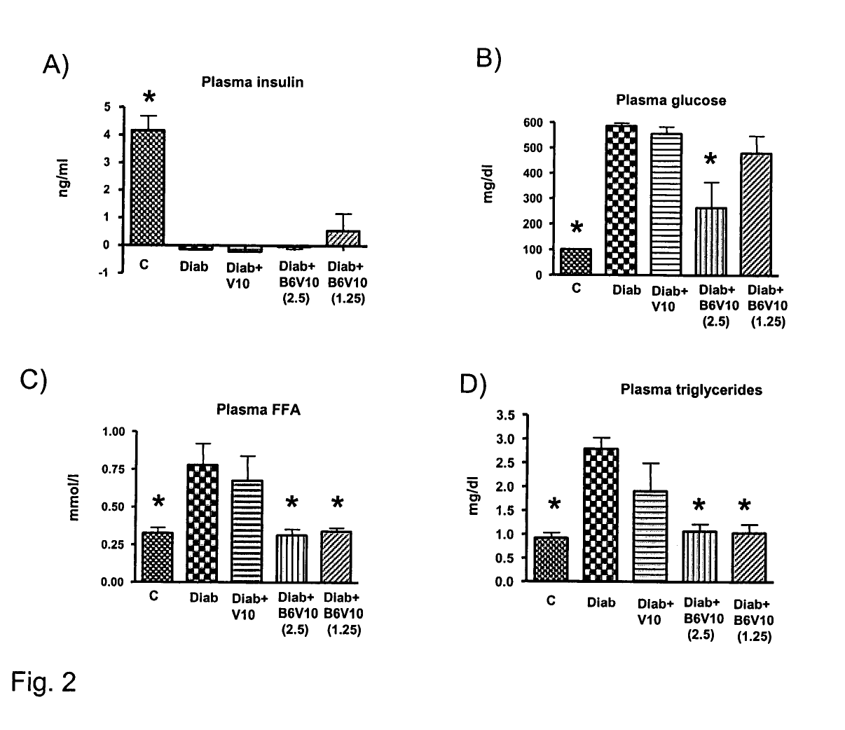

Subcutaneous administration of hexaquis(benzylammonium) decavanadate was tested to determine whether it ameliorated the dyslipidemia associated with diabetes. Diabetes was induced in rats by intravenous administration of a large dose of streptozotocin (100 mg/kg), causing a complete disappearance of plasma insulin and very high concentrations of plasma glucose (FIGS. 2A and 2B). Induction of diabetes was also associated with substantial increases in plasma free fatty acid and triglyceride concentrations compared to nondiabetic rats (FIGS. 2C and 2D). Under these conditions, subcutaneous treatment with different doses of hexaquis(benzylammonium) decavanadate normalized plasma concentrations of free fatty acids and triglycerides so that no differences between control and hexaquis(benzylammonium) decavanadate-treated rats were detectable (FIGS. 2C and 2D). Under these conditions, treatment with decavanadate did not alter plasma concentration of triglycerides or free fatty acids (FIGS. 2C and 2D).

### Example 3

### Effect of Subcutaneous and Chronic Administration of Hexaquis(benzylammonium) Decavanadate on Plasma Cholesterol Levels in Diabetic Rats

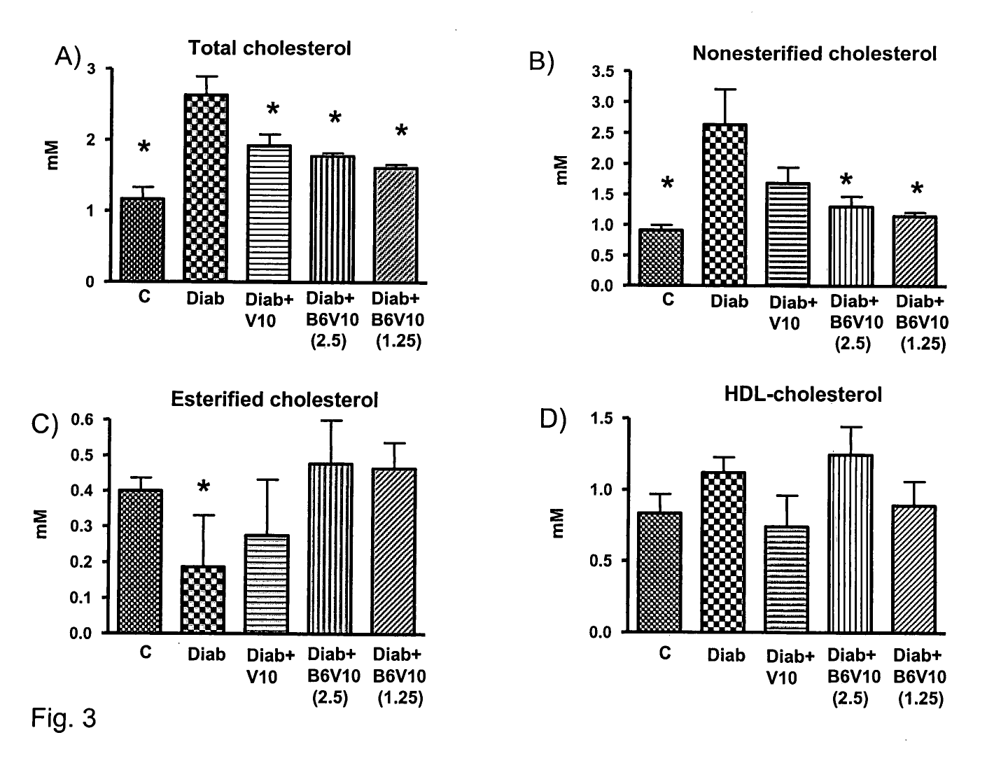

Subcutaneous administration of hexaquis(benzylammonium) decavanadate was performed to determine the effect on altered plasma concentrations of cholesterol associated to diabetes. Diabetes was induced in rats by intravenous administration of a large dose of streptozotocin (100 mg/kg). Experimentally-induced Diabetes was associated with substantial increases in total plasma cholesterol and nonesterified cholesterol and a reduction in esterified cholesterol concentrations compared to nondiabetic rats (FIG. 3A). HDL-cholesterol concentrations were unaltered by diabetes (FIG. 3B). Under these conditions, subcutaneous treatment with different doses of hexaquis(benzylammonium) decavanadate reduced plasma concentrations of total cholesterol and nonesterified cholesterol and increased esterified cholesterol concentrations so no differences between control and hexaquis(benzylammonium) decavanadate-treated rats were detectable (FIG. 3C). Under these conditions, treatment with decavanadate also caused a significant reduction in total cholesterol but levels of nonesterified and esterified cholesterol were not significantly different than in untreated diabetic rats (FIG. 3D).

It should be understood that the foregoing disclosure emphasizes certain specific embodiments of the invention and that all modifications or alternatives equivalent thereto are within the spirit and scope of the invention as set forth in the appended claims.

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims. To particularly point out and distinctly claim the subject matter regarded as invention, the following claims conclude this specification.

We claim:

1. An antidyslipidemic formulation comprising a pharmaceutically acceptable amine, or a pharmaceutically acceptable salt of said amine, that is a substrate for semicarbazide-sensitive amine oxidase or other copper-containing amine oxidase, in admixture with pharmaceutically acceptable excipients or carriers.

2. An antidyslipemic formulation of claim 1, further comprising a pharmaceutically acceptable vanadium (IV)/(V) compound.

3. An antidyslipidemic formulation of claim 2, wherein the amine and the vanadium compound are formulated for simultaneous, separate or sequential administration.

4. An antidyslipdemic formulation according to claims 2 or 3, wherein the vanadium compound is a vanadate, peroxovanadium complex, vanadyl salt or vanadyl complex.

5. An antidyslipdemic formulation according to claim 4, wherein the vanadium compound is a vanadate.

6. An antidyslipdemic formulation according to claim 5, wherein the vanadium compound is sodium orthovanadate.

7. An antidyslipdemic formulation according to claims 1, 2, or 3, wherein the amine is benzylamine, tyramine, 3-phenyl-propylamine, 2-(4-fluoro-phenyl)-ethylamine, 4-phenyl-butylamine, 4-fluoro-benzylamine, 2,3-dimethoxybenzylamine, 1-naphtalenemethylamine, deoxyepinephrine, epinephrine, norepinephrine, dopamine, histamine, β-phenylethylamine, N-acetylputrescine, tryptamine, n-octylamine, n-pentylamine, kynuramine, 3-methoxytyramine, or n-decylamine, hexylethanolamine, octopamine, spermine, spermidine, N-acetylspermine, or N-acetylspermidine.

8. An antidyslipdemic formulation according to claim 7, wherein the amine is benzylamine, tyramine, 3-phenyl-propylamine, 2-(4-fluoro-phenyl)-ethylamine, 4-phenyl-butylamine, 4-fluoro-benzylamine, 2,3-dimethoxybenzylamine, or 1-naphtalenemethylamine.

9. An antidyslipdemic formulation according to claim 7, wherein the amine is 3-phenyl-propylamine, 2-(4-fluoro-phenyl)-ethylamine, 4-phenyl-butylamine, 4-fluoro-benzylamine, 2,3-dimethoxybenzylamine, or 1-naphtalenemethylamine.

10. A method for treating or preventing dyslipidemia in an animal by administering a pharmaceutically acceptable amine or a pharmaceutically acceptable salt of said amine that is a substrate of semicarbazide-sensitive amine oxidase or other copper-containing amine oxidase.

11. A method according to claim 10, further comprising the step of administering a pharmaceutically acceptable vanadium (IV)/(V) compound.

12. A method according to claim 11, wherein the amine and the vanadium compound are administered simultaneously, separately or sequentially.

13. A method according to claims 10, 11 or 12, wherein the vanadium compound is a vanadate, peroxovanadium complex, vanadyl salt or vanadyl complex.

14. A method according to claim 13, wherein the vanadium compound is a vanadate.

15. A method according to claim 13, wherein the vanadium compound is sodium orthovanadate.

16. A method according to claims 10, 11, or 12, wherein the amine is tyramine, benzylamine, 3-phenyl-propylamine, 2-(4-fluoro-phenyl)-ethylamine, 4-phenyl-butylamine, 4-fluoro-benzylamine, 2,3-dimethoxybenzylamine, 1-naphtalenemethylamine, deoxyepinephrine, epinephrine, norepinephrine, dopamine, histamine, β-phenylethylamine, N-acetylputrescine, tryptamine, n-octylamine, n-pentylamine, kynuramine, 3-methoxytyramine, or n-decylamine, hexylethanolamine, octopamine, spermine, spermidine, N-acetylspermine, or N-acetylspermidine

17. A method according to claim 16, wherein the amine is tyramine, benzylamine, 3-phenyl-propylamine, 2-(4-fluoro-phenyl)-ethylamine, 4-phenyl-butylamine, 4-fluoro-benzylamine, 2,3-dimethoxybenzylamine, or 1-naphtalenemethylamine.

18. A method according to claim 16, wherein the amine is 3-phenyl-propylamine, 2-(4-fluoro-phenyl)-ethylamine, 4-phenyl-butylamine, 4-fluoro-benzylamine, 2,3-dimethoxybenzylamine, or 1-naphtalenemethylamine.

19. A pharmaceutical composition containing a pharmaceutically acceptable amine, or a pharmaceutically acceptable salt of said amine, that is a subtrate for semicarbazide-sensitive amine oxidase or other copper-containing amine oxidase, and a pharmaceutically-acceptable excipient, for the treatment and/or prevention of dyslipidemia.

20. A pharmaceutical composition of claim 19, further comprising a pharmaceutically acceptable vanadium (IV)/(V) compound.

21. A pharmaceutical composition of claim 20, comprising a combined preparation of the amine and the vanadium-containing compound, for simultaneous, separate or sequential administration.

22. A pharmaceutical composition according to claims 20 or 21, wherein the vanadium compound is a vanadate, peroxovanadium complex, vanadyl salt or vanadyl complex.

23. A pharmaceutical composition according to claim 22, wherein the vanadium compound is a vanadate.

24. A pharmaceutical composition according to claim 22, wherein the vanadium compound is sodium orthovanadate.

25. A pharmaceutical composition according to claims 19, 20, or 21, wherein the amine is tyramine, benzylamine, 3-phenyl-propylamine, 2-(4-fluoro-phenyl)-ethylamine, 4-phenyl-butylamine, 4-fluoro-benzylamine, 2,3-dimethoxybenzylamine, 1-naphtalenemethylamine, deoxyepinephrine, epinephrine, norepinephrine, dopamine, histamine, β-phenylethylamine, N-acetylputrescine, tryptamine, n-octylamine, n-pentylamine, kynuramine, 3-methoxytyramine, or n-decylamine, hexylethanolamine, octopamine, spermine, spermidine, N-acetylspermine, or N-acetylspermidine.

26. A pharmaceutical composition according to claim 25, wherein the amine is tyramine, benzylamine, 3-phenyl-propylamine, 2-(4-fluoro-phenyl)-ethylamine, 4-phenyl-butylamine, 4-fluoro-benzylamine, 2,3-dimethoxybenzylamine, or 1-naphtalenemethylamine.

27. A pharmaceutical composition according to claim 25, wherein the amine is 3-phenyl-propylamine, 2-(4-fluoro-phenyl)-ethylamine, 4-phenyl-butylamine, 4-fluoro-benzylamine, 2,3-dimethoxybenzylamine, or 1-naphtalenemethylamine.

28. An antidyslipidemic formulation according to claims 1, 2, or 3, wherein the amine is a compound or a pharmaceutically acceptable solvate thereof, including hydrates, for preparing a medicament for treating or preventing a lipid disorder in a mammal, including a human, wherein the compound has the formula: 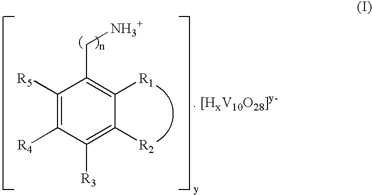
wherein
R₁, R₂, R₃, R₄ and R₅ are radicals that are independently H, OH, (C₁-C₆)-alkyl, (C₁-C₆)-alkoxy, NR₆R₇, (CH₂)ₚNR₈R₉, O(CH₂)_{q}-phenyl, CONR₁₀R₁₁, COR₁₂, CF₃, OCF₃, F, Cl, Br, NO₂, or CH₂NHC(═NH)NH₂; or alternatively R₁ and R₂ are bound together forming a ring with a fused benzene;
p and q are integers from 1 to 3;
R₆, R₇, R₈, R₉, R₁₀, R₁₁ and R₁₂ are radicals that are independently H, (C₁-C₄)-alkyl or phenyl or other aryl; and
where n is an integer from 1 to 3;
where x is an integer from 0 to 2, and
where y is an integer from 4 to 6,
provided that x+y=6.

29. An antidyslipidemic formulation according to claim 28, wherein R₁, R₂, R₃, R₄ and R₅ are radicals that are independently H, (C₁-C₆)-alkyl, OH, (C₁-C₆)-alkoxy, O(CH₂)_{q}Ph, CF₃, OCF₃, F, Cl, Br, and NO₂; or wherein R₁ and R₂ are bound together forming a ring with a fused benzene in the compound of formula (I).

30. An antidyslipidemic formulation according to claim 28, wherein n=1 in the compound of formula (I).

31. An antidyslipidemic formulation according to claim 28, wherein x=0 and y=6 in the compound of formula (I).

32. An antidyslipidemic formulation according to claim 31, wherein the compound of formula (I) is hexaquis(benzylammonium) decavanadate (V) dihydrate.

33. An antidyslipidemic formulation according to claim 28, wherein x=1 and y=5 in the compound of formula (I).

34. An antidyslipidemic formulation according to claim 33, wherein the compound of formula (I) is hexaquis(benzylammonium) decavanadate (V) dihydrate.

35. An antidyslipidemic formulation according to claim 22, wherein x=2 and y=4 in the compound of formula (I).

36. An antidyslipidemic formulation according to claim 35, wherein the compound of formula (I) is hexaquis(benzylammonium) decavanadate (V) dihydrate.
